(19) United States
(12) Patent Application Publication
(10) Pub. No.: US 2023/0220351 A1
(43) Pub. Date: Jul. 13, 2023

(54) LARGE-SCALE PRODUCTION OF EXOSOMES FROM PRIMED MESENCHYMAL STROMAL CELLS FOR CLINICAL USE

(71) Applicant: BOARD OF REGENTS, THE UNIVERSITY OF TEXAS SYSTEM, Austin, TX (US)

(72) Inventors: Elizabeth SHPALL, Houston, TX (US); Katy REZVANI, Houston, TX (US); Mayela MENDT, Houston, TX (US)

(21) Appl. No.: 18/000,414

(22) PCT Filed: Jun. 24, 2021
(86) PCT No.: PCT/US2021/070772

§ 371 Date: Dec. 1, 2022

Related U.S. Application Data
(60) Provisional application No. 63/043,328, filed on Jun. 24, 2020.

(51) Int. Cl. C12N 5/077; C12N 5/073; A61K 35/28

(52) U.S. Cl. CPC C12N 5/0669; C12N 5/0605; A61K 35/28; C12N 2501/24; C12N 2501/25; C12N 2501/2301; C12N 2501/2317; C12N 2510/00

(57) ABSTRACT

Embodiments of the disclosure encompass systems, methods, and compositions for producing exosomes from primed mesenchymal stem cells that are expanded in the presence of IFNγ, TNFα, IL-1β, and IL-17. The systems, methods, and compositions ay occur in an automated cell expansion system that allows for controllable parameters and from which cells and exosomes may be harvested at one or more times as part of a particular regimen. In specific embodiments, the exosomes may be provided to an individual in need thereof, including in some cases when the exosomes comprise one or more therapeutic agents.

This application claims priority to U.S. Provisional Patent Application Ser. No. 63/043,328, filed Jun. 24, 2020, which is incorporated by reference herein in its entirety.

### TECHNICAL FIELD

Embodiments of the disclosure concern at least the technical fields of cell biology, molecular biology, cell expansion systems and components, and medicine.

### BACKGROUND

Clinical options for treatment of disease and delivery of therapeutic agents are always in demand and in need of improvement. The present disclosure satisfies needs in the art by providing a reliable, reproducible, and practical system for producing exosomes as a means for therapy and/or for therapeutic agent delivery.

### BRIEF SUMMARY

The present disclosure is directed to systems, methods, and compositions for production and use of exosomes. In particular embodiments, the disclosure concerns systems, methods, and compositions for production of exosomes for the purpose of being used as part of a treatment, including as part of a therapeutic agent for use for an individual in need thereof, and/or as part of a delivery agent itself to deliver one or more therapeutic agents to an individual in need thereof. In certain embodiments, exosomes are produced from particular cells using multiple agents in the production method of the exosomes. In specific aspects, exosomes are produced from particular cells in the presence of multiple proteins, such as in a culture medium, and in specific embodiments at least 1, 2, 3, 4, or more of the proteins are cytokines. Such exosomes may be produced from particular cells, including at least stem cells, and for example, mesenchymal stromal cells (MSCs, which may also be referred to as mesenchymal stem cells). The MSCs may be derived from any suitable tissue, but in a specific case they are derived from umbilical cord tissue.

Particular embodiments of the disclosure encompass the production of exosomes from umbilical cord tissue-derived MSCs using a combination of cytokines including IFNγ, TNFα, IL-1β, and IL-17. The exosomes produced by this method are utilized for treatment of one or more medical disorders, including at least immune disorders. In such cases, the exosomes may or may not carry one or more therapeutic agents for one or more specific medical conditions.

Embodiments of the disclosure provide for systems that utilize one or more particular parameters for the process of producing specific exosomes from MSCs that have been expanded in the presence of IFNγ, TNFα, IL-1β, and IL-17, and therefore are primed. Such a system may be automated and in specific embodiments utilizes hollow fibers having surfaces onto which the MSCs adhere during the expansion process and concomitant exposure to IFNγ, TNFα, IL-1β, and IL-17.

Embodiments of the disclosure include methods of producing exosomes from mesenchymal stromal cells (MSCs, including from umbilical cord tissue, bone marrow, adipose tissue, dental tissue, placental tissue, or a mixture thereof), comprising the steps of (a) culturing MSCs in the presence of an effective amount of interferon (IFN)γ, tumor necrosis factor (TNF)α, interleukin (IL)-1β, and IL-17; and (b) collecting the exosomes from the culture. Steps (a) and (b) may or may not occur more than once. Steps (a) and (b) may occur 2, 3, 4, 5, 6, 7, 8, 9, 10, or more times, in some cases. In specific cases, step (b) occurs more than once and the collecting occurs in intervals of about 48 hours.

In any method encompassed herein, a culturing step may occur for at least 18 hours or for 18-24 hours. In any method encompassed herein, a collecting step may occur once or multiple times including with a duration between collecting steps being about 1 day, 2 days, 3 days, 4 days, or longer. In specific embodiments, exosomes collected at different times comprise substantially the same genotype and/or phenotype. In any method encompassed herein, a culturing step occurs in the presence of specific concentrations or conditions of CO₂ (such as about 5%), O₂ (such as about 20%, and/or the culturing step occurs under conditions balanced with nitrogen.

In certain embodiments with respect to the exosomes, the exosomes comprise higher levels of one or more immunosuppressive factors compared to exosomes produced from culture that does not comprise IFNγ, TNFα, IL-1β, and IL-17. In some cases, the exosomes comprise HLA-G, PD-L1, IL-10, TGF-β, IDO, and PD-L2. In specific cases, the exosomes comprise higher levels of one or more of HLA-G, PD-L1, IL-10, TGF-β, IDO, and PD-L2 compared to exosomes produced from culture that does not comprise IFNγ, TNFα, IL-1β, and IL-17. In specific embodiments, the exosomes comprise the markers CD9, CD63, CD47, and/or CD81. In particular cases, the exosomes have enhanced control of T cell proliferation compared to exosomes produced from culture that does not comprise IFNγ, TNFα, IL-1β, and IL-17.

In particular embodiments, the method occurs in an automated system, including a system configured to comprise continuous perfusion of medium through at least part of the system. The system may be closed or semi-closed. The method may or may not occur in a bioreactor, including one with multiple hollow fibers. One or more surfaces inside a bioreactor may be modified to allow adherence of cells, including one or more surfaces inside the bioreactor being modified to comprise one or more extracellular matrix proteins, including at least fibronectin, for example.

Specific embodiments of the disclosure include methods comprising the step of extracting a sample from the system, and the sample may or may not be tested for one or more characteristics of the exosomes. In specific embodiments, any step of the method may or may not utilize media that lacks platelet lysate. In a method referred to herein, step (b) utilizes media that lacks platelet lysate, in some cases. In specific cases, any step of the method may or may not utilize media that comprises L-alanyl-L-glutamine dipeptide. In a method referred to herein, step (b) utilizes media that comprises L-alanyl-L-glutamine dipeptide, in some cases. The culture in step (a) referred to herein may further comprise media that comprises L-alanyl-L-glutamine dipeptide. The culture in step (a) may further comprise alpha MEM media, heparin, human platelet lysate and L-alanyl-L-glutamine dipeptide.

Embodiments of the disclosure include delivering an effective amount of the exosomes to an individual in need thereof. In some cases following delivery to an individual in need thereof, the exosomes have enhanced migration to peripheral tissue (brain, bone marrow, kidney, spleen, or a combination thereof, for example) compared to exosomes produced from culture that does not comprise IFNγ, TNFα, IL-1β, and IL-17. Any exosomes may directly or indirectly regulate an innate immune response or adaptive immune response in the individual in need thereof. In some embodiments, an individual in need thereof has an immune disorder (autoimmune disorder or an alloimmune disorder), cancer, heart disease, kidney disease, lung disease, liver disease, infection, or a combination thereof. In specific embodiments, the immune disorder is graft-versus-host disease.

In certain embodiments, the exosomes are modified, including prior to delivery to an individual. In specific embodiments, the exosomes are exo-fucosylated before delivery to an individual in need thereof. In some embodiments, the exosomes are transduced or transfected with a fucosyl transferase to facilitate removal of surface fucosyl groups, allowing enhanced uptake by cells. The exosomes may be loaded to comprise one or more therapeutic agents, including at least loaded by a vector, electroporation, transfection, using a cationic liposome transfection agent, or a combination thereof. One or more therapeutic agents may be miRNA, siRNA, shRNA, protein (antibody or antibody fragment or antibody conjugate or mixture thereof), peptides, drug, lipids, DNA, RNA, or a combination thereof.

Embodiments of the disclosure encompass exosomes produced from any one of the methods encompassed herein, compositions comprising the exosomes, and pharmaceutical compositions comprising the exosomes. The exosomes may further comprise one or more additional therapeutic agents.

Embodiments of the disclosure include methods of treating an individual for an immune disorder, cancer, heart disease, kidney disease, lung disease, liver disease, infection, or a combination thereof, comprising the step of administering to the individual a therapeutically effective amount of exosomes produced by any method encompassed herein. The immune disorder may be an alloimmune disorder or an autoimmune disorder. In some cases, the method further comprises administering to the individual a second therapy for the respective immune disorder, cancer, heart disease, kidney disease, lung disease, liver disease, infection, or a combination thereof. The MSCs may be autologous or allogeneic with respect to the individual. The exosomes may be administered via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir. In some embodiments, the exosomes are administered in conjunction with at least one additional therapeutic agent.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1D describe one example of a procedure designed to produce extracellular vesicles (EVs), such as exosomes, primed from MSC using a bioreactor, such as the Terumo Cell Expansion System (Terumo BCT®; Lakewood, Colo.).

FIGS. 2A-2B show that umbilical cord MSCs produce higher levels of exosomes than Bone Marrow MSCs. FIG. 2A) Bar graph showing the number of exosomes per cell per day produced by clinical grade BM MSCs versus umbilical cord tissue (CBt) MSCs. (n=3) FIG. 2B) Representative histogram of flow cytometry of unprimed CBt MSC-exosomes and primed CBt MSC-exosomes showing the expression of typical exosome markers as CD63, CD81, CD9, and CD47 (red, right peaks) in comparison with the isotype (blue, left peaks).

FIG. 3 shows that primed human Umbilical Cord MSC-derived exosomes express on their surfaces higher levels of immunosuppressive factors than unprimed. Representative histogram of flow cytometry of unprimed CBt MSC-exosomes (blue) and primed CBt MSC-exosomes (red, at least right peaks) showing the expression of typical exosome markers as CD63, CD81, CD9, and CD47.

FIG. 4 demonstrates that primed umbilical cord MSC (UCMSC)-derived exosomes modulate T cell proliferation and secretion in vitro in a dose dependent manner. Representative histograms of the secretion of inflammatory by stimulated T cells are provided.

FIG. 5 demonstrates that primed CBt-MSC-derived exosomes show superior properties to control T cell proliferation in vitro compared to unprimed exosomes.

FIGS. 6A-6C show that primed CBt-derived exosomes demonstrate efficacy for treating GVHD. Infusion of primed CBt-MSC-derived exosomes (8 μg/animal) 2 times per week increases the overall survival rate (FIG. 6A), reduces the lost weight (FIG. 6B) and the clinical signs of GVHD (FIG. 6C) in a xenograft graft-versus-host disease (GVHD) mice model.

FIGS. 7A-7B show biodistribution of pre-labeled activated UCMSC-derived exosomes injected into mice. Fluorescence of DIR-labeled MSC exosomes 48 hours after intravenous administration of 5×10⁹ labeled exosomes in NSG mice. (FIG. 7A) Dissected organs. (FIG. 7B) Dissected lung, bone (femur), brain and liver.

FIG. 8 shows that modification of proteins on the surface of MSC-derived exosomes from different sources affect their uptake by human umbilical vein endothelial cells (HUVEC).

FIG. 9A-9C show flow cytometry of BMMSC and cord tissue MSCs (CBtiMSCs)-derived exosomes non-transduced and transduced with FT-6 after 48 h of transduction. (FIG. 9A) Representative scatter plot of CD63 expression versus cell-surface fucosylation, sLe^{X} (HECA-452) expression on the surface of exosomes derived from bone marrow MSCs non-transduced (blue, mostly upper left), bone marrow MSCs transduced with the enzyme FT-6 (red, mostly upper right), using as control the isotype (grey, mostly bottom left), analyzed by flow cytometry. (FIG. 9B) Representative scatter plot and mean fluorescent intensity of CD63 expression versus cell-surface fucosylation, SLeX (HECA-452) expression on the surface of exosomes derived from cord blood tissue MSCs non-transduced (blue, mostly upper left), bone marrow MSCs transduced with the enzyme FT-6 (red, mostly upper right), using as control the isotype (grey, mostly lower left), analyzed by flow cytometry. (FIG. 9C) Graph Bar of the mean fluorescent intensity (MFI) of FT6 transduced BMMSC derived exosomes (left) and CbtiMSCs derived exosomes collected at 6H 24H and 48H after transduction. In the triplet of bars, from left to right they represent 6H, 24H, and 48H.

FIGS. 10A-10B concern fucosylation of e-selectin ligands on the surface proteins of BMMSC and CBtiMSC-derived exosomes enhance their uptake by HUVEC. (FIG. 10A) Representative scatter plot showing the uptake of prelabeled (DiR) BMMSCs exosomes from nontransduced (middle panel) and FT-6 transduced (right panel) at 6H, 24H, and 48H of coculture with HUVEC, using as control HUVEC non incubated with exosomes (left panel), and analyzed by flow cytometry. (FIG. 10B) Representative scatter plot showing the uptake of prelabeled (DiR) CBtiMSCs exosomes from nontransduced (middle panel) and FT-6 transduced (right panel) at 6H, 24H, and 48H of coculture with HUVEC, using as control HUVEC non incubated with exosomes (left panel), and analyzed by flow cytometry.

FIG. 11 shows uptake of CFSE-pre labeled exosomes from non-transduced and FT-6 transduced CbtiMSCs by GSC 8-11 glioblastoma cells line labeled with m-Cherry after 1 hour of incubation. Representative scatter plot showing the uptake of prelabeled (CFSE) CBtiMSCs exosomes from nontransduced and FT-6 transduced at 1H of coculture with GSC 8-11 (mCherry), using as control GSC 8-11 non incubated with exosomes and analyzed by flow cytometry.

### DETAILED DESCRIPTION

### I. Examples of Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. The terms “about”, “substantially” and “approximately” mean, in general, the stated value plus or minus 5%.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

The term “therapeutically effective amount” refers to an amount sufficient to produce a desired therapeutic result, for example an amount of exosomes sufficient to improve at least one symptom of a medical condition in a subject to whom the cells are administered.

The term “subject” or “patient” or “individual” refer to either a human or non-human, such as primates, mammals, and vertebrates. In particular embodiments, the subject is a human. The subject is of any age, gender, or race.

The term “treatment” refers to a therapeutic intervention that ameliorates a sign or symptom of a disease or pathological condition. Prevention of a disease does not require a total absence of disease. For example, a decrease of at least 50% can be sufficient. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” or “effective” as used throughout this application refers to anything that promotes or enhances the well-being of the subject with respect to the medical treatment of this condition. This includes, but is not limited to, a reduction in the frequency or severity of the signs or symptoms of a viral infection and associated disease or medical condition.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The present disclosure describes specific systems, methods, and compositions to produce exosomes from MSCs. As demonstrated herein, MSC-primed exosomes prepared according to the disclosed procedures are stable and bioactive. The exposure of MSCs to a cocktail of inflammatory cytokines for a particular length of time, for example 18-24 hours, during their ex vivo expansion enriches for highly immunosuppressive population of MSCs and stimulates the secretion of primed exosomes, which in particular embodiments are loaded with 1, 2, or several immunosuppressive factors. CBt-MSCs produce significantly higher numbers of exosomes compared with bone marrow (BM)-derived MSCs, in at least some embodiments. Phenotypic characterization confirmed that CBt-MSC and BM-MSC-derived exosomes express the same levels of the exosome markers CD9, CD63, CD47 and CD81 (as examples of markers). Functionally, CBt-MSC-derived exosomes, primed or not, controlled cell proliferation and secretion by T cells stimulated in vitro in a dose dependent manner. Furthermore, flow cytometry studies established that CBT-MSC-primed exosomes contained higher levels of PDL-1, PDL-2, HLA-G, TGF-β and IL-10 than the unprimed CBT-MSC exosome counterparts. Additionally, biodistribution studies in a xenogeneic mouse mice model revealed that CBT-MSC-primed exosomes migrate more efficiently to peripheral tissues such as, brain, bone marrow, kidney, and spleen persisting longer (more than 48 hours) than unprimed exosomes. The established method is practical, efficient, and allows for the clinical use of CBt-MSC-primed exosomes as therapeutic agents for the treatment of individuals, such as with alloimmune or autoimmune disorders, or as vehicles for gene and drug delivery and as therapeutic agents for regenerative medicine settings, for example.

### II. Systems and Methods of Producing Exosomes

The present disclosure provides systems and methods of producing extracellular vesicles (EVs) as exosomes. In specific cases, the present disclosure concerns a novel, good manufacturing practice (GMP)-compliant strategy to produce exosomes. The exosomes are produced under particular conditions in combination with being produced from particular cells. Thus, in particular embodiments, exosomes are produced from MSCs that have been subjected to one or more particular cytokines. In certain embodiments, the exosomes are produced from MSCs of any kind that have been primed by one or more specific cytokines and, in particular cases, multiple cytokines.

In specific embodiments, exosomes are produced from MSCs that are expanded (proliferated) in the presence of multiple and specific cytokines. Specific methods allow for the production of exosomes from mesenchymal stem cells (MSCs), comprising the step of culturing the MSCs in an effective amount of IFNγ, TNFα, IL-1β, and IL-17 in the culture such that the MSCs expand (proliferate) and naturally secrete the exosomes into the culture for subsequent collection.

The exosomes produced from the MSCs may come from primed MSCs subjected to IFNγ, TNFα, IL-1β, and IL-17, in particular embodiments. The MSCs are exposed to the cytokines and, as a result, produce exosomes having particular one or more suitable characteristics. The disclosed methods provide for production of higher numbers of clinical grade primed extravesicles as exosomes, carrying high levels of immunosuppressive factors and which migrate more efficiently to peripheral tissues when compared to unprimed CBt-MSCs or marrow-derived MSCS.

In particular embodiments, the MSCs are from umbilical cord tissue, but they can come from any source including, but not limited to, bone marrow, adipose tissue, dental and placental tissue.

In particular embodiments of the disclosure, the process of the disclosure produces exosomes from primed cells. As used herein, “primed” refers to cells (and exosomes produced therefrom) that have been exposed to a particular cytokine regimen (IFNγ, TNFα, IL-13, and IL-17). The primed exosomes refer to exosomes secreted by cells that have been exposed to the particular cytokine regimen. In specific embodiments, the exosomes are not further exposed to any cytokines other than IFNγ, TNFα, IL-1β, and IL-17. Any media in which the MSCs are cultured may comprise, consist of, or consist essentially of IFNγ, TNFα, IL-1β, and IL-17 with respect to cytokines.

Any step in the process may have a particular media, duration of time, presence of one or more particular gases at specific concentrations, presence or absence of movement (such as rotation), and a combination thereof, for example. In particular embodiments, there is sequential exposure of MSCs of any kind to a cocktail of multiple inflammatory cytokines that are used for the “priming” of the cells. The cells are incubated with media supplemented with the cytokine regimen for a particular amount of time, in some cases, to produce activated cells. This is followed by washing and collection of the cells and exosomes secreted from the activated cells. The collection of the exosomes (that may be referred to herein as harvesting) may include one step or multiple steps; in cases when the collection of the exosomes occurs more than once, there may or may not be an interval of time by which the exosomes are collected, such as about 12, 18, 24, 36, 48, 60, 72, or more hours between collections.

The media in which the cells and exosomes are collected may be of a particular kind, and in specific steps when the cells and exosomes are collected the media lacks platelet lysate (PLT-free). In specific cases, the cells are primed over the course of about 22 hours, and then cells are washed and exosomes secreted from the activated cells are collected approximately every 48 hours in the EC media PLT-free (the EC media-PLT free may or may not comprise alpha MEM media supplemented with 2 mM of Glutamax (synthetic reagent similar to L-glutamine and that comprises L-alanyl-L-glutamine dipeptide)). These sequential steps may be repeated, such as repeated for a total of 2, 3, 4, or more times.

In specific embodiments, the suspension of cells and exosomes are harvested from the system under conditions in which the exosomes produced from the cells consistently have the same or substantially the same markers and physiology. Thus, in specific cases at different times of harvesting, the exosomes are the same or substantially the same by their majority of exosomes having one or more of the same expression markers. In some cases, a suspension of cells and exosomes are harvested on one or more days following initiation of a cell priming step, such as day 9, 12, 15, 17, 20, and/or 23 following initiation of the cell priming step(s). In some embodiments, a suspension of cells and exosomes are harvested at a certain time period following the cells being incubated with media supplemented with one or more particular cytokines, such as being harvested within about 24, 48, or 72 hours following exposure of the cells to the media supplemented with one or more particular cytokines.

In particular embodiments, the process to produce the exosomes occurs in a bioreactor, although in alternative cases it does not. In specific embodiments, part or all of the process occurs in a bioreactor having controllable conditions that in specific cases may be automated. Although the bioreactor may be of any kind, in specific embodiments the bioreactor comprises a hollow fiber system that may or may not comprise one or more pathways. The multiple hollow fibers comprise inner surfaces suitable for adherence of cells or suitable for modification such that cells may adhere to them, in particular embodiments. Alternative systems utilize the WAVE Bioreactor™ (GE Healthcare) or the G-Rex® system (Wilson Wolf), as examples.

In certain embodiments, the hollow fiber bioreactor may be a functionally closed (or semi-closed) system designed for a large-scale cell culture of adherent or non-adherent cells. The system allows the cells to grow (expand in number) in a dynamic environment allowing the continuous perfusion of medium that under suitable conditions mimics particular in vivo intravascular and extravascular compartments in at least some bioreactors. That is, in specific cases an intravascular compartment is configured to mimic the intravascular region of the blood system and/or an extravascular compartment is configured to mimic the extravascular hematopoietic system. The hollow fiber system in specific cases comprises hundreds or thousands of semi-permeable pores for the culture of desired cells, including adherent cells. Membranes may make up the inner walls of the hollow fibers and allow exchange of gas and/or nutrients with a homogenous approach, maximizing the growth rate of the cells in a short time. In particular embodiments, the process is specifically designed to be suitable for growth of MSCs and to allow for the collection of the exosomes secreted by the cells in a customized method.

Components of the bioreactor system comprise vessels and/or compartments for introducing media and/or cells to the system, vessels and/or compartments for expanding the cells (and thereby produce exosomes from the expanding/expanded cells), and vessels and/or compartments for harvesting the cells, the conditioned media comprising the exosomes, and so forth. Examples of compartments for any part of the system include a cell inlet bag, media bag, harvest bag, and waste bag, in specific embodiments. The bioreactor system utilizes thousands of semi-permeable hollow fibers onto which the cells are adherent, either naturally or because the hollow fibers in the system have been manipulated to allow for adherence of the desired cells. In specific embodiments, the system also comprises a gas regulator (that may be referred to as a gas transfer module) that stabilizes desired gas concentrations in the media. Such a gas regulator allows for, if desired, continual infusion of one or more gases into the bioreactor. In specific embodiments, the process to produce the desired exosomes utilizes well-defined concentrations of CO₂ (for example, about 5%) O₂ (for example, about 20%) and nitrogen (for example, the conditions are nitrogen balanced).

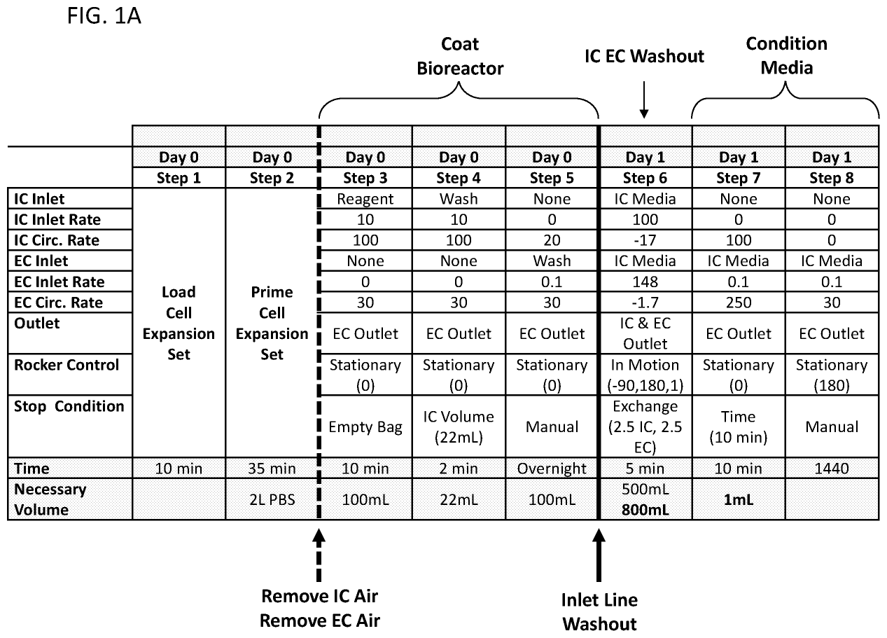

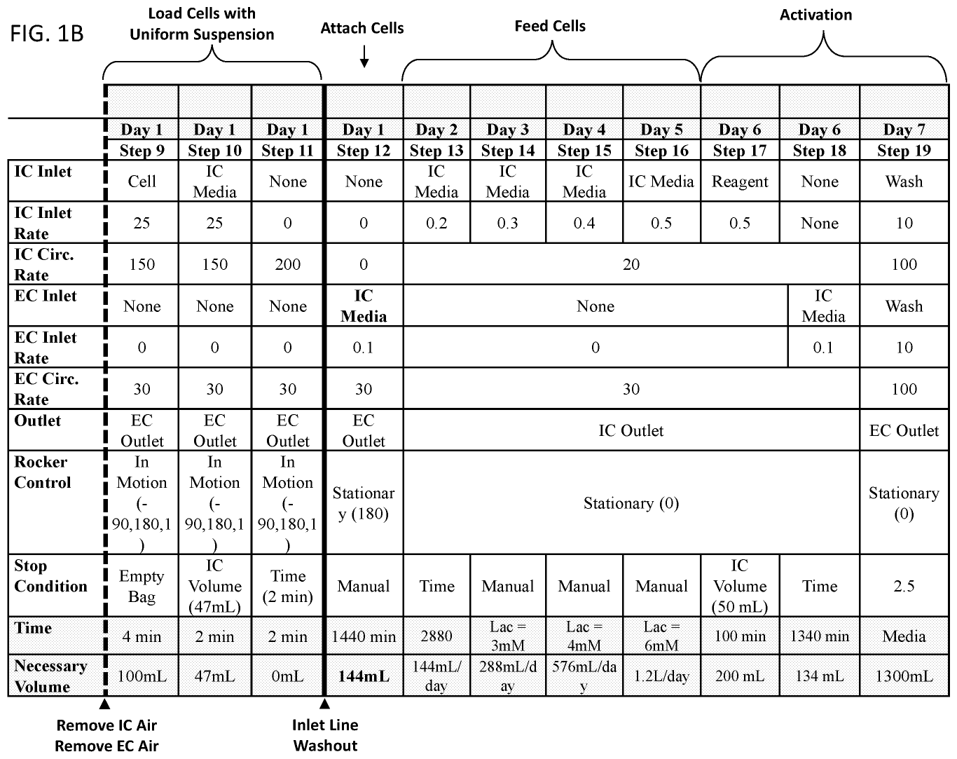

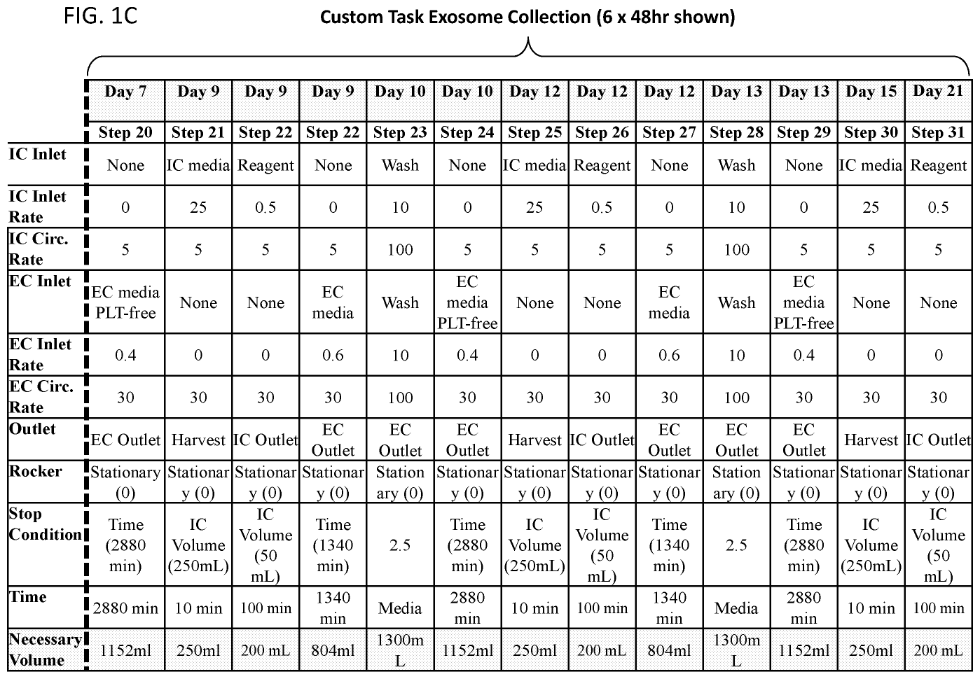

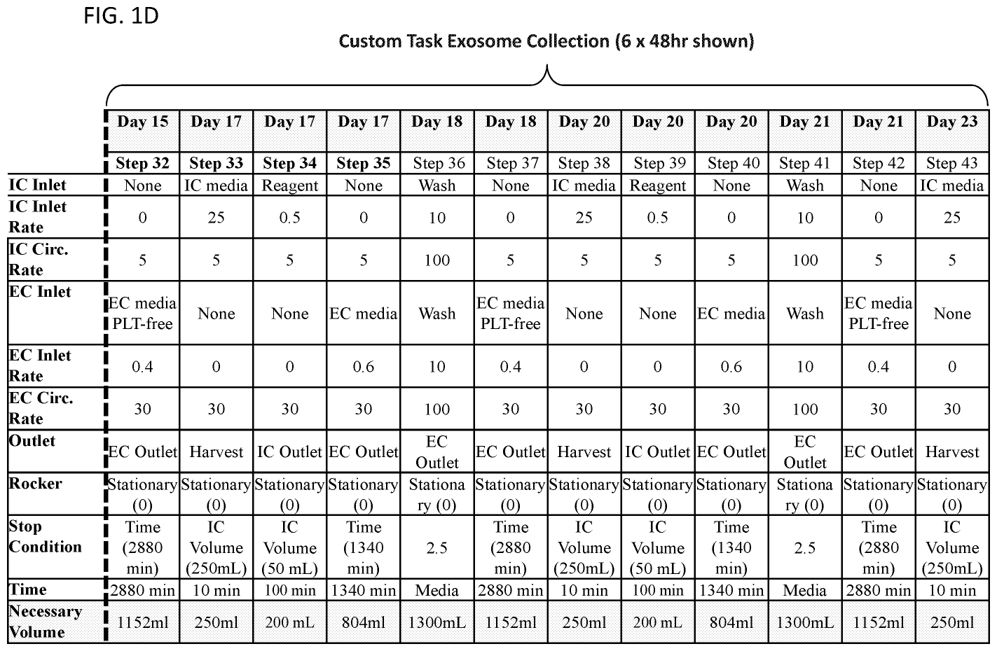

FIGS. 1A-1D provide one example of a procedure and system for producing exosomes. In specific cases of the system, there may be an intracapillary (IC) pathway and/or an extracapillary (EC) pathway. In cases wherein the bioreactor comprises an IC and/or EC pathway, they may be maintained by inlet pumps that determine the flow of new medium into each side of the bioreactor, and circulation pumps that determine the rate at which the medium in each side of the bioreactor is moved through its circuit. In particular embodiments for the bioreactor system, a hollow fiber bioreactor system is utilized that is formed by micropores and is divided into separate intracapillary (IC) and extracapillary (EC) fluid pathways. In specific embodiments, the fluidics for the IC and EC pathways are maintained by inlet pumps that determine the flow of new medium into each side of the bioreactor, and circulation pumps that determine the rate at which the medium in each side of the bioreactor is moved through its circuit. In specific embodiments, the cells are seeded onto intracapillary compartments, whereas the EC compartment is used to feed the cells with media.

Generally speaking, appropriate steps are taken to prepare the system prior to loading of the cells, such as preparation of the physical components of the system to facilitate expansion of the cells. The system may be closed or may be semi-closed (as used herein, refers to during the production of exosomes some steps require the opening of the system and the exposure of the sample to the air). Prior to subjecting the cells to be expanded to the system, the bioreactor may be subjected to one or more components and/or one or more conditions to facilitate adherence of cells to the bioreactor. Cell media may be loaded into the system prior to loading of the cells.

For adherent cell production, cells attach and proliferate on the inner surface of each fiber. Suspended cells can be flushed, leaving the adherent cell production for expansion. Automated cell feeding and waste removal means may be part of the system, in specific embodiments. In at least some cases, sampling of cells/conditioned media from the system may be provided for without or with interruption of the process. In particular embodiments, after cell expansion the adherent cells are released from the hollow fiber walls into suspension, and the suspension including cells and exosomes secreted therefrom are collected.

FIG. 1A demonstrates specific beginning steps that may be employed in a process for generation of desired exosomes. Day 0 in an example of a process may comprise Steps 1, 2, 3, 4, 5, or all 5 of the first 5 Steps. In such a case, Step 1 may comprise a Load Cell Expansion Set Step. In specific embodiments, the “Load Cell Expansion Set” step refers to the installation of the disposable cells expansion sets containing the hollow fiber bioreactors (where the cells will grow) onto a Quantum® cell expansion system and the connection of all the lines that allows the supply of CO₂ (for example, 5%), medium, air, and the outline for the waste. During Step 2 of the process is the “Prime Cell Expansion Set” Step, in which the whole hollow fiber system is filled through the inlet and outline connections with phosphate-buffered saline (PBS) without Ca²⁺ and Mg²⁺, removing the air.

During Steps 3 to 5, the bioreactor may be coated prior to loading of the cells, including coating within the hollow fibers of the system. In some cases, in Step 3 a reagent (for example, the mix of IL-17, IFN-γ, TNFα, and IL-1β diluted in alpha MEM media comprising 2 mM L-alanyl-L-glutamine dipeptide) is applied as part of steps for coating a bioreactor. In some cases the bioreactor following application of the reagent is washed (for example, with a buffer such as PBS). In specific cases during Steps 3 to 5, a membrane surface of an intracapillary compartment (e.g., a tubing) of the bioreactor is coated with one or more compounds to promote cell adherence in the bioreactor. In specific cases, the hollow fibers of the bioreactor are coated with human fibronectin (or any extracellular matrix-type reagent, such as retronectin) to promote cell adherence. In specific cases, the fibronectin is an extracellular matrix protein (that may be obtained commercially) from human plasma, for example.

In some embodiments, Steps 6-8 occur on Day 1. In some cases, Step 6 concerns washing out of the IC and EC pathways (and may entail motion of the sets of the system, including at −90, 180, and 1 degrees of movement of the hollow fiber set)), and Steps 7-8 concern addition of conditioned media to the system (and may have stationary sets of the system). In each of Steps 6-8, IC Media is applied to the EC inlet, but in Step 6 IC Media is also applied to the IC inlet, in some cases. In certain embodiments, IC media comprises a source of basic growth media, heparin, platelet lysate, and L-glutamine or a similar compound. In specific embodiments, human platelet lysate is utilized because it is a xenogeneic-free, human allogenic replacement for fetal bovine serum, which contains several growth factors useful for cell growth (e.g., epidermal growth factor, platelet derived growth factor, IL-6, insulin-like growth factor, or a combination thereof) and is obtained from human blood platelets after freeze/thaw cycles. In specific embodiments, IC media comprises alpha MEM media supplemented with Heparin 2 U/mL, 5% human platelet lysate (hPLT), and 2 mM of Glutamax.

FIG. 1B shows examples of other Steps in Day 1 through Steps in at least part of Day 7 in this example of a process for exosome production. Steps 9-11 concern loading of the cells into the system to produce a uniform suspension in the system. For example, in Step 9, cells may be input into the system through the IC inlet, followed by an appropriate volume of IC media (Step 10); in Step 11, the IC circulation rate may be increased. In each of Steps 9-11, the expansion set may be subjected to motion, such as at −90, 180, and 1. In Step 12, the cells may be allowed to attach upon ceasing motion of the rocker and allowing stationary conditions to support adherence of the cells within the hollow fibers of the sets. Step 12 includes input of IC media to the EC inlet, in particular embodiments.

Days 2-5 may include Steps 13-16, respectively, of the process in which the cells are allowed to expand, including in a stationary setting. IC Media is provided through the IC inlet, and in specific cases the IC Inlet Rate is gradually increased over the course of Steps 13-16. In specific embodiments, IC Media is not input into the EC Inlet in Steps 13-17. In Step 17, reagent (mix of four cytokines (IL-17, IFNγ, TNFα, and IL-1β) diluted in alpha MEM media containing 2 mM of Glutamax) is added into the IC inlet instead of IC Media. In Step 18, no IC Media or reagent are input into the IC inlet, although IC Media may be input into the EC Inlet. Step 19 on Day 7 includes a wash step, e.g., with a buffer such as PBS.

FIG. 1C shows examples of Steps 20-31 across the course of Days 7-15, in specific aspects. In Step 20, EC media that is platelet-free is input into the EC Inlet, and in the following Step 21 the suspension is harvested following input of IC media into the IC inlet. The Reagent added at Steps 22, 26, and 31 and on Days 17 and 20 is a mix of four cytokines (IL-17, IFNγ, TNFα, and IL-1β) diluted in alpha MEM media containing 2 mM of Glutamax. Harvesting steps may continue periodically thereafter, such as every day, every 2 days, every 3 days, every 4 days, every 5 days, and so on (see also FIG. 1D).

In certain embodiments, the cells are exposed to IL-17, IFN-γ, TNFα, and IL-1B in the process at the same time or at substantially the same time. The concentration of each of IL-17, IFNγ, TNFα, and IL-1β may be a particular concentration or range of concentrations in the media for the cells. In specific embodiments, IFNγ and TNFα may have a concentration range in the media from about 10 ng/ml to about 100 ng/ml. In specific embodiments, IL-17 in the media may utilize a range between about 5 ng/ml and about 30 ng/ml, and a media concentration of IL-1β may be about 5 ng/ml to about 50 ng/ml.

Once the cells are harvested from the process, including from the system in such cases, the exosomes may be separated by any suitable means from the supernatant and cells. In some cases, there are multiple harvests from the process, and the supernatant, cells, and exosomes from the process may be pooled prior to any further separation or modification steps. In certain cases, exosomes from multiple harvests are processed separately and combined later.

In some embodiments, the exosomes are enriched or concentrated following the production process. As one example, the exosomes are separated from cells, cell fragments, and/or larger or smaller vesicles through physical and/or chemical means. In specific cases, the exosomes are concentrated through one or more centrifugations, one or more filtrations (such as ultrafiltration and/or diafiltration), one or more chemical precipitation, size exclusion chromatography, microfluidics, or a combination thereof. Different centrifugation steps may occur at different speeds, and/or different filtration steps may occur at different sizes.

The exosomes may be used immediately or substantially immediately, or they may be stored prior to use, for example at −80° C. or in liquid nitrogen.

In some embodiments, the exosomes are concentrated prior to modification of any kind, whereas in other cases the exosomes are modified prior to concentration. The exosomes may be analyzed following the production process, following the concentration step, and/or during the process itself. Such analysis includes identifying one or more markers, identifying size, determining concentration, determining one or more specific activities for the exosomes (such as migration or immunosuppression, and/or anti-T cell activity) or a combination thereof.

### III. Modification of Exosomes

Although the exosomes comprise one or more certain characteristics or activities as a result of being produced from MSCs (including particular MSCs, such as from umbilical cord tissue) and/or as a result of being produced from MSCs expanded in the presence of IL-17, IFNγ, TNFα, and IL-1β, the exosomes may be further modified. In particular cases, the exosomes are further modified to harbor (carry) one or more therapeutic agents. In some cases, the exosomes themselves are transfected or transduced with one or more therapeutic agents or agents or “pay loads” that target several different cancers, or siRNA or miRNA that also target cancers that express the relevant target genes. The transfection or transduction agents may also enhance any other activities of the exosomes, including, for example, uptake of the exosomes into desired cells or reduction in the inflammation produced by the cells once the exosomes are taken up by those cells. Examples include cytokine genes such as TNFα, IL10 and/or Indoleamine-pyrrole 2,3-dioxygenase (IDO), as well as miRs such as miR-124, all known to reduce inflammation. In specific embodiments, the one or more therapeutic agents is miRNA, siRNA, shRNA, proteins (including antibodies of any kind), peptides, drug, lipids, DNA, RNA, or a combination thereof. In specific embodiments, the exosomes are modified such that they express a fucosyl transferase (such as FT-6 and/or FT-7) to allow de-fucosylation prior to use. In at least some cases, the de-fucosylation allows or facilitates the exosomes to be taken up or incorporated into endothelial cells, immune cells, or cancer cells.

The modification of the exosomes may occur by any suitable method in the art, but in specific cases the exosomes are loaded with one or more therapeutic agents by a vector, electroporation, transfection, using a cationic liposome transfection agent, or a combination thereof.

The therapeutic agent(s) loaded into the exosomes in particular embodiments are exogenous with respect to the MSCs. They can be introduced into the exosomes by a number of different techniques. In particular embodiments of the disclosure, the exosomes are loaded by electroporation or the use of a transfection reagent. Electroporation conditions may vary depending on the charge and size of the therapeutic agent(s). Typical voltages are in the range of 20V/cm to 1000V/cm, such as 20V/cm to 100V/cm with capacitance typically between 25 μF and 250 μF, such as between 25 μF and 125 μF. A voltage in the range of 150 mV to 250 mV, particularly a voltage of 200 mV may be used for loading exosomes with therapeutic agent(s) according to the present disclosure.

In some embodiments, the exosomes may be loaded with therapeutic agent(s) using one or more transfection reagents. Particular transfection reagents for use in accordance with the present disclosure include cationic liposomes.

In another embodiment, exosomes may also be loaded by transforming or transfecting the MSCs with a nucleic acid construct that expresses the therapeutic agent(s), such that the therapeutic agent(s) are present in the exosomes as the exosomes are produced from the cell.

In specific embodiments, the exosomes are of a specific size such that their size determines the type of therapeutic agents that they can carry. In particular cases, the exomes are 30-400 nm in size, including 30-350, 30-300, 30-250, 30-200, 30-150, 30-100, 30-50, 50-400, 50-350, 50-300, 50-250, 50-200, 50-150, 50-100, 100-400, 100-350, 100-300, 100-250, 100-200, 200-400, 200-350, 200-300, 200-250, 250-400, 250-350, 250-300, 300-400, 300-350, or 350-400 nm in size. In specific embodiments, the exosomes are able to be loaded with any type of therapeutic agent(s), such as proteins (including antibodies or fragments thereof), peptides, lipids, short RNA sequences and/or short DNA sequences (each less than about 1000 nucleotides), lipids, miR, anti-miR, siRNA, shRNA, and/or drugs, including small molecule drugs. The therapeutic agent(s) may be cancer therapeutic agents, therapeutic agents for microbial infection, therapeutic agents for heart disease, therapeutic agents for lung disease, therapeutic agents for liver disease, therapeutic agents for kidney disease, therapeutic agents for neurological disease, or a combination thereof. For cancer therapeutic agents, the agent(s) may be a drug, small molecular, antibody, inhibitory RNA targeting an oncogene, tumor suppressor protein, or a combination or mixture thereof.

In some embodiments, the exosomes comprise one or more antibodies or antibody fragments. The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. Each light chain is comprised of a light chain variable region (abbreviated herein as V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR). An antibody of use in the invention may be a monoclonal antibody or a polyclonal antibody, and will preferably be a monoclonal antibody. An antibody of use in the invention may be a chimeric antibody, a CDR-grafted antibody, a nanobody, a human or humanized antibody or an antigen binding portion of any thereof. For the production of both monoclonal and polyclonal antibodies, the experimental animal is typically a non-human mammal such as a goat, rabbit, rat or mouse but may also be raised in other species such as camelids. The term “antigen-binding portion” of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv antibodies are also intended to be encompassed within the term “antigen-binding portion” of an antibody. These antibody fragments may be obtained using conventional techniques known to those of skill in the art, and the fragments may be screened for utility in the same manner as intact antibodies. An antibody of use in the invention may be a human antibody or a humanized antibody.

In some embodiments, the exosomes are loaded with one or more cancer drugs, including one or more chemotherapies, such as the examples of paclitaxel, doxorubicin, adriamycin, gemcitabine, cisplatin, bortezomib, palbociclib, ibrutinib, nivolumab, pegfilgrastim, filgrastim, bevacizumab, trastuzumab, rituximab, lenalidomide, Herceptin, taxol, and combinations thereof.

In certain embodiments, the exosomes are loaded with one or more antimicrobial agents (an antimicrobial agent may be a natural or synthetic substance that kills or inhibits the growth of microorganisms or pathogens, such as bacteria, fungi, algae, or viruses). The antimicrobial agents may be an antibiotic, antifungal, antiviral, and so forth.

In alternative embodiments, the exosomes are not loaded with a therapeutic drug but instead are loaded with one or more gene-modifying components, such as that comprise a CRISPR-Cas system, including a specific guide RNA and an endonuclease.

### IV. Exosome Compositions

In particular embodiments, exosomes produced by the process of the disclosure may comprise a particular genotype and/or phenotype. In specific embodiments, the exosomes produced by the disclosed methods are of a particular genotype and/or phenotype because they were produced from cells primed with exposure to IL-17, IFNγ, TNFα, and IL-1β. In specific cases, priming of MSCs with IL-17, IFNγ, TNFα, and IL-1 (3 produce exosomes that comprise one or more anti-inflammatory molecules. The exosomes have activity for direct or indirect regulation of innate and adaptive immune responses, in particular embodiments, including because they were produced from the primed cells. In specific embodiments, the exosomes comprise anti-inflammatory activity that exosomes that were not produced from the primed cells lack, because they were not produced from the primed cells.

In specific embodiments, the exosomes produce one or more of Programmed Death-ligand 1 (PD-L1), human leukocyte antigen G (HLA-G), interleukin 10 (IL-10), Transforming growth factor beta (TGF-β). As a result, the exosomes themselves comprise anti-inflammatory activity even in conditions wherein they lack any therapeutic agent(s) that they could hold. The ability of the exosomes to comprise PD-L1, HLA-G, IL-10, and TGF-β gives them activity in which they can directly or indirectly regulate innate and adaptive immune responses in an individual, particularly when compared to exosomes that have not been derived from primed cells and even in the absence of therapeutic agent(s).

In some embodiments, the produced exosomes are manipulated to carry one or more therapeutic agents. Exosomes produced by the disclosed methods are small extracellular vesicles of 50-400 nm, and their size will dictate the size of therapeutic agent(s) that they can hold. In particular embodiments, the exosomes are 50-400, 50-350, 50-300, 50-250, 50-200, 50-150, 50-100, 100-400, 100-350, 100-300, 100-250, 100-200, 100-150, 150-400, 150-350, 150-300, 150-250, 150-200, 200-400, 200-350, 200-300, 200-250, 250-400, 250-350, 250-300, 300-400, 300-350, or 350-400 nm. In specific embodiments, the exosomes are limited to carry small molecules, such as proteins, lipids, short RNA and DNA sequences, and drugs, including small molecule drugs.

In some embodiments, the exosome compositions comprise one or more therapeutic agent(s), such as proteins (including antibodies or fragments thereof), peptides, lipids, short RNA sequences, short DNA sequences, lipids, and/or drugs, including small molecule drugs. The therapeutic agent(s) may be cancer therapeutic agents, therapeutic agents for microbial infection, therapeutic agents for heart disease, therapeutic agents for lung disease, therapeutic agents for liver disease, therapeutic agents for kidney disease, therapeutic agents for neurological disease, or a combination thereof. The exosomes may comprise antibodies, in specific cases. The exosomes may comprise one or more antimicrobial agents or may comprise one or more gene modifying components.

### V. Methods of Using Exosomes

In particular embodiments, exosomes are useful for the treatment of one or more medical conditions. The exosomes may be used for the systemic or local delivery of therapeutic compounds.

In some cases, the exosomes are useful for one or more immune disorders. In specific embodiments, exosomes derived from umbilical cord tissue (CBt-MSC)-derived MSCs are useful for the treatment of immune disorders and for the systemic delivery of therapeutic compounds for the immune disorders. Methods and compositions of the disclosure allow for generation of a large scale of activated exosomes from CBt-MSCs, carrying immunomodulatory factor(s) that can be used for the treatment of any inflammatory disorder, regenerative therapies, and as carrier vehicles for the delivery of different factors, including at least miR, anti-miR, siRNA, and therapeutic drugs.

The exosomes of the disclosure may be used for treatment of any medical condition for which modulation of an innate immune response or adaptive immune response may be beneficial. In specific cases, this is a result of the exosomes secreting the anti-inflammatory molecules of PD-L1, HLA-G, IL-10, and TGF-β as a result of being produced from the primed MSCs described herein.

Any individual with an inflammatory disorder, as well as disorders where gene or drug therapy could be delivered, may benefit from exosomes produced from methods of the disclosure. The inflammatory disorder may comprise inflammation as one of its symptoms, but there could be other symptoms. The inflammation may be acute or chronic. Examples of inflammatory disorders include at least asthma, rheumatoid arthritis, inflammatory bowel diseases, gout, atherosclerosis, chronic peptic ulcer, tuberculosis, rheumatoid arthritis, periodontitis, ulcerative colitis and Crohn's disease, sinusitis, active hepatitis, and so forth.

Embodiments of the disclosure include methods for treatment of heart disease of any kind, including at least coronary artery disease, heart failure, cardiomyopathy, valvular heart disease, arrhythmia, genetic defects of the heart, and so forth.

Embodiments of the disclosure include methods for treatment of lung disease, such as pulmonary hypertension, asthma, bronchopulmonary dysplasia (BPD), allergy, cystic fibrosis, Chronic Obstructive Pulmonary Disease, idiopathic pulmonary fibrosis, acute respiratory distress syndrome (ARDS), pneumonia, pleural effusion, and so forth.

Embodiments of the disclosure include methods for treatment of a microbial infection of any kind, including a pathogenic infection. The infection may be bacterial, viral, fungal, or protozoan. Examples of bacterial include, but are not limited to, Actinomyces, Bacillus, Bacteroides, Bordetella, Bartonella, Borrelia, Brucella, Campylobacter, Capnocytophaga, Chlamydia, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea polypeptides, Salmonella, Shigella, Staphylococcus, group A streptococcus, group B streptococcus, Treponema, and Yersinia. Examples of fungi include, but are not limited to, Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha. Examples of protozoa include, but are not limited to, Babesia, Balantidium, Besnoitia, Cryptosporidium, Eimeria, Encephalitozoon, Entamoeba, Giardia, Hammondia, Hepatozoon, Isospora, Leishmania, Microsporidia, Neospora, Nosema, Pentatrichomonas, Plasmodium. Examples of helminth parasites include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca Spirometra, Stephanofilaria, Strongyloides, Strongylus, Thelazia, Toxascaris, Toxocara, Trichinella, Trichostrongylus, Trichuris, Uncinaria, Wuchereria, Pneumocystis, Sarcocystis, Schistosoma, Theileria, Toxoplasma, and Trypanosoma. Examples of viruses include adenovirus, alphavirus, calicivirus, coronavirus (including SARS-CoV, SARS-CoV-2, and MERS), distemper virus, Ebola virus, enterovirus, flavivirus, hepatitis virus, herpesvirus, infectious peritonitis virus, leukemia virus, Marburg virus, Norwalk virus, orthomyxovirus, papilloma virus, parainfluenza virus, the, paramyxovirus, parvovirus, pestivirus, picorna virus, pox virus, rabies virus, reovirus polypeptides, retrovirus, rotavirus, and vaccinia virus.

In certain embodiments, the exosomes are utilized for individuals in need of regeneration of tissue for any reason. The tissue in need of regeneration may be of any kind, but in specific embodiments the tissue is brain, lung, spleen, liver, heart, kidney, pancreas, intestine, testis, and bone. The exosomes in such cases are therapeutic at least in part because they are suitable to migrate in the individual. In particular embodiments, the exosomes produced by methods encompassed herein are useful as regenerative therapies to target organs including brain, lung, spleen, liver, heart, kidney, pancreas, intestine, testis, and bone, as examples of target tissues.

The exosome compositions of the disclosure may be administered by any suitable means. Administration to a human or animal subject may be selected from parenteral, intramuscular, intracerebral, intravascular (including intravenous), subcutaneous, intranasal, intracardiac, intracerebroventricular, intraperitoneal or transdermal administration.

The exosomes may be delivered as a composition. The composition may be formulated for any suitable means of administration, including parenteral, intramuscular, intracerebral, intravascular (including intravenous), intracardiac, intracerebroventricular, intraperitoneal, subcutaneous, intranasal or transdermal administration. Compositions for parenteral administration may include sterile aqueous solutions which may also contain buffers, diluents and other suitable additives. The exosomes of the disclosure may be formulated in a pharmaceutical composition, which may include pharmaceutically acceptable carriers, thickeners, diluents, buffers, preservatives, and other pharmaceutically acceptable carriers or excipients and the like in addition to the exosomes.

A “pharmaceutically acceptable carrier” (excipient) is a pharmaceutically acceptable solvent, suspending agent or any other pharmacologically inert vehicle for delivering one or more nucleic acids to a subject. Typical pharmaceutically acceptable carriers include, but are not limited to, binding agents (e.g. pregelatinised maize starch, polyvinylpyrrolidone or hydroxypropyl methylcellulose, etc.); fillers (e.g. lactose and other sugars, microcrystalline cellulose, pectin, gelatin, calcium sulfate, ethyl cellulose, polyacrylates or calcium hydrogen phosphate, etc.); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc); disintegrates (e.g. starch, sodium starch glycolate, etc.); or wetting agents (e.g. sodium lauryl sulphate, etc.).

The compositions provided herein may additionally contain other adjunct components conventionally found in pharmaceutical compositions. Thus, for example, the compositions may contain additional compatible pharmaceutically-active materials or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions provided herein.

A therapeutically effective amount of composition is administered. The dose may be determined according to various parameters, especially according to the severity of the condition, age, and weight of the patient to be treated; the route of administration; and the required regimen. A physician will be able to determine the required route of administration and dosage for any particular patient. Optimum dosages may vary depending on the relative potency of individual constructs, and can generally be estimated based on EC50s found to be effective in vitro and in in vivo animal models. In general, dosage is from 0.01 mg/kg to 100 mg per kg of body weight. A typical daily dose is from about 0.1 to 50 mg per kg, preferably from about 0.1 mg/kg to 10 mg/kg of body weight, according to the potency of the specific construct, the age, weight and condition of the subject to be treated, the severity of the disease and the frequency and route of administration. Different dosages of the construct may be administered depending on whether administration is by intramuscular injection or systemic (intravenous or subcutaneous) injection. In some cases, the dose of single or multiple systemic injections is in the range of 10 to 100 mg/kg of body weight.

In some cases, the individual may have to be treated repeatedly, for example once or more daily, weekly, monthly or yearly. Persons of ordinary skill in the art can easily estimate repetition rates for dosing based on measured residence times and concentrations of the construct in bodily fluids or tissues. Following successful treatment, it may be desirable to have the individual undergo maintenance therapy, wherein the construct is administered in maintenance doses, ranging from 0.01 mg/kg to 100 mg per kg of body weight, once or more daily, to once every 20 years.

### EXAMPLES

The following examples are included to demonstrate particular embodiments of the disclosure. It should be appreciated by those of skill in the art that the techniques disclosed in the examples that follow represent techniques discovered by the inventor(s) to function well in the practice of the methods and compositions of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the disclosure.

### Example 1

### Generation of Activated CBT-MSC-Derived Exosomes for Clinical Use

The present examples provides a novel, robust, GMP-compliant strategy to produce extracellular vesicles as exosomes from activated umbilical cord tissue-derived mesenchymal stromal cells (CBt-MSCs) using a Cell Expansion System (Bioreactor) and a cocktail of cytokines (IFNγ, TNFα, IL-1β, and IL-17). This approach allowed for the consistent generation of between 1.0×10⁹-1.5×10⁹ clinical grade primed CBt-MSC-exosomes per passage in a very short period of time, which comprise one or more immunomodulatory factors such as TGF-B, IDO, PD-L1, PD-L2, HLA-G, IL10, and higher levels than chemokine receptors than unprimed CBt-MSC-exosomes. A useful step in the generation of these exosomes is the priming with cytokines in the bioreactor itself, in specific concentrations and sequence, minimizing microbial contamination and maximizing the numbers and immunosuppressive as well as regenerative potency in this GMP-compliant process. The choice of cytokines, their dose and schedule when added to the continuously-perfused bioreactor confer potency and other useful aspects of the method. This new protocol efficiently incorporates the sequential exposure of CBt-MSCs to the cocktail of inflammatory cytokines for 24 hours during their ex vivo expansion and subsequent collection of an enriched highly immunosuppressive population of CBTtMSC-exosomes (primed), which improve their therapeutic effects.

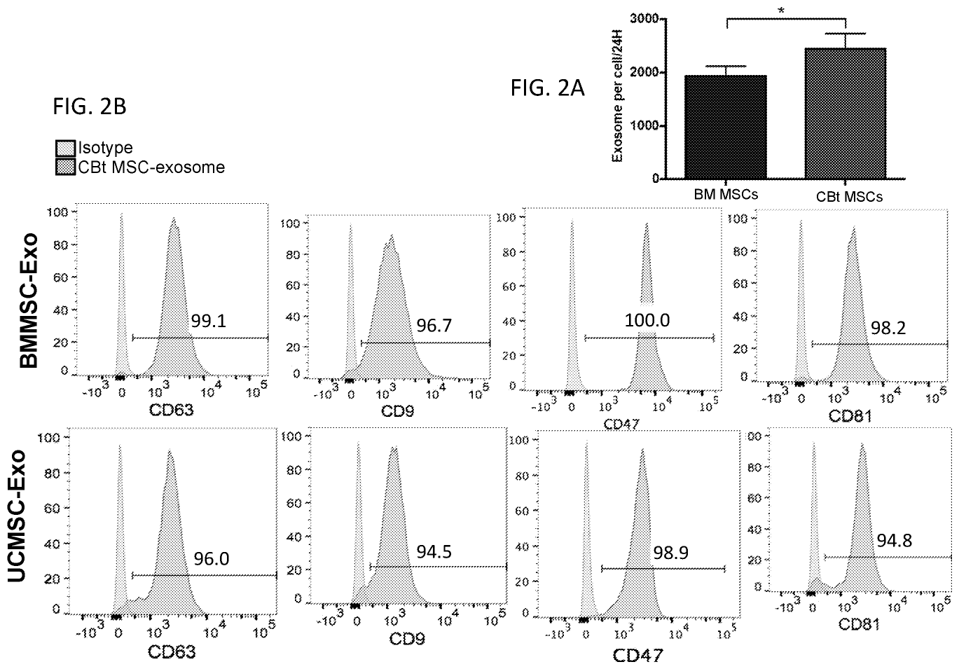

In specific cases to generate these exosomes, the bioreactor is seeded at a density of approximately 450 cells/cm² with approximately 5.0×10⁷ CBt-derived MSCs (although any suitable range may also include 2.0×10⁷ to 5.0×10⁷); the cells are cultured for 6 days in alpha MEM medium supplemented with L-glutamine plus human platelet lysate (PLT) in 5% oxygen. Once the MSCs in the bioreactor reach ≥85% confluence, one can pre-activate the cells with the cocktail of pro-inflammatory cytokines which includes: IFNγ (10 ng/ml), TNFα (10 ng/ml), IL17 (10 ng/ml) and IL-1β (10 ng/ml) for 24 hours. The cells are then washed and the growth media exchanged for serum free media. The medium is left in the bioreactor for 48 hours (conditioned media) and then collected for exosome purification. The steps of activation, washing and exchange of media are sequentially repeated every 48 hours for a total of 6 harvests (FIG. 1). After 23 days, the conditioned media that had been collected is pooled and filtered in a semi-closed system, and the extracellular vesicles as exosomes isolated by ultracentrifugation at 100,000 g for 4 hours at 4° C., using XE-90 Ultracentrifuge (Beckman Coulter). The purified exosome identity is confirmed by the flow cytometric expression of the exosome surfaces markers: CD63, CD47, CD9 and CD81 (FIG. 2).

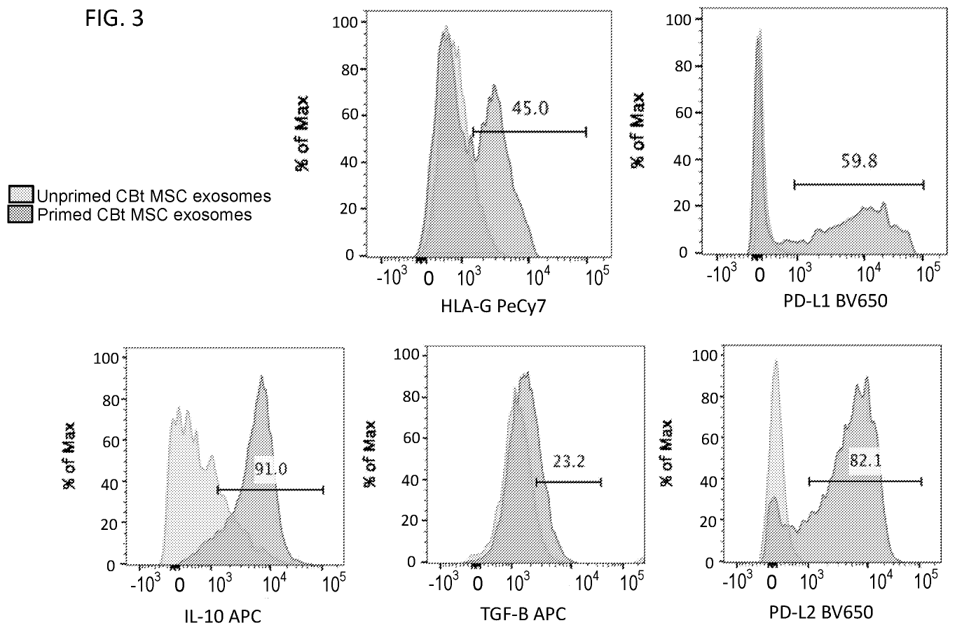

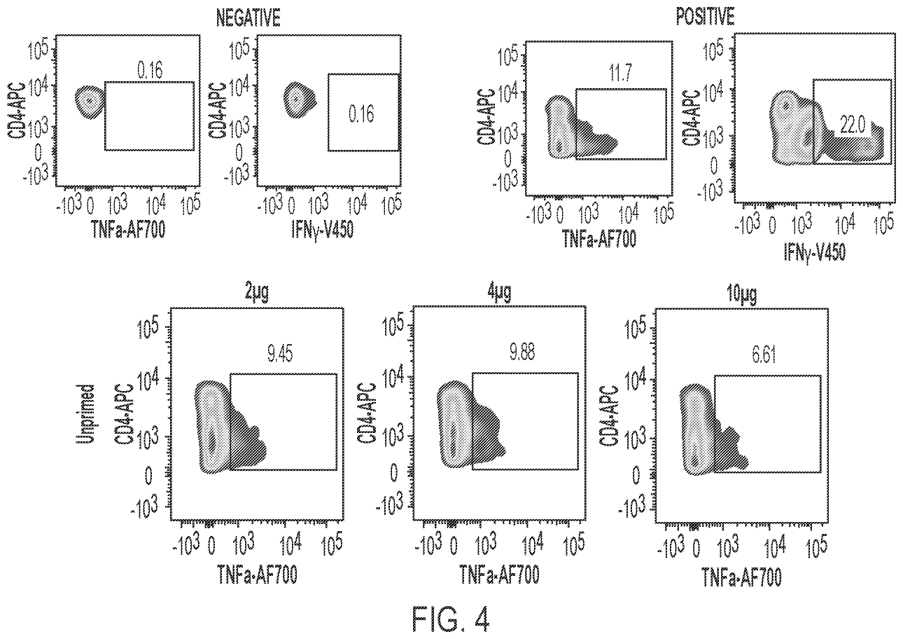

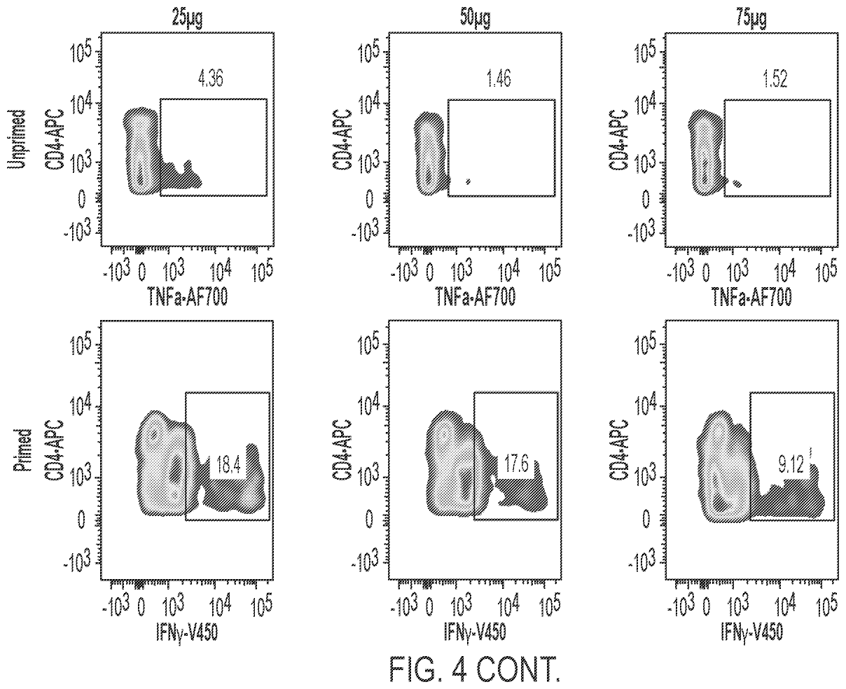

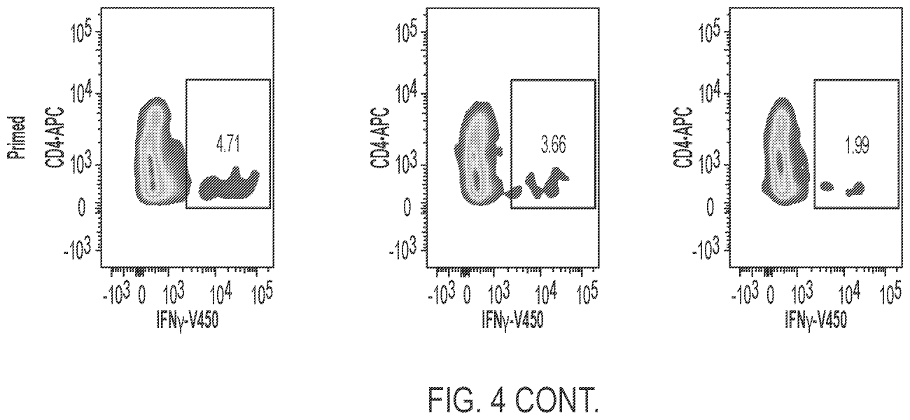

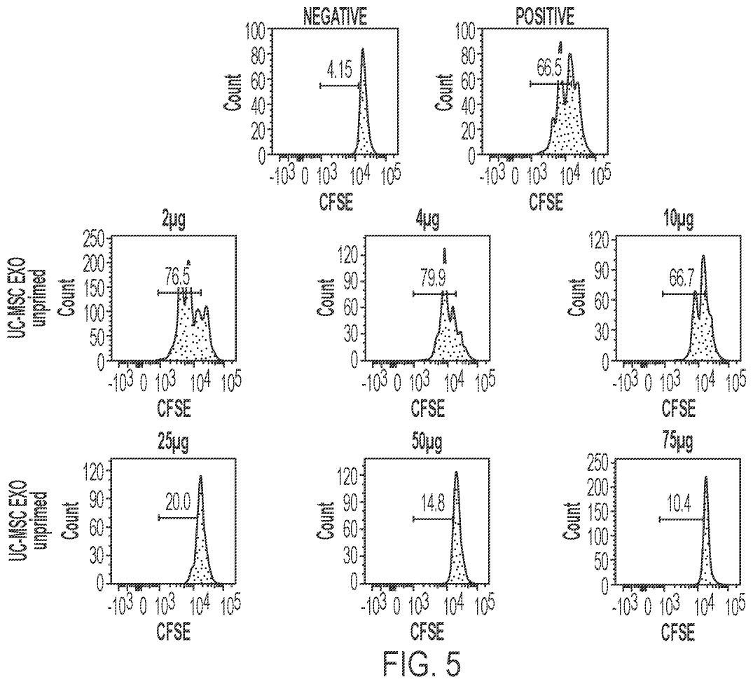

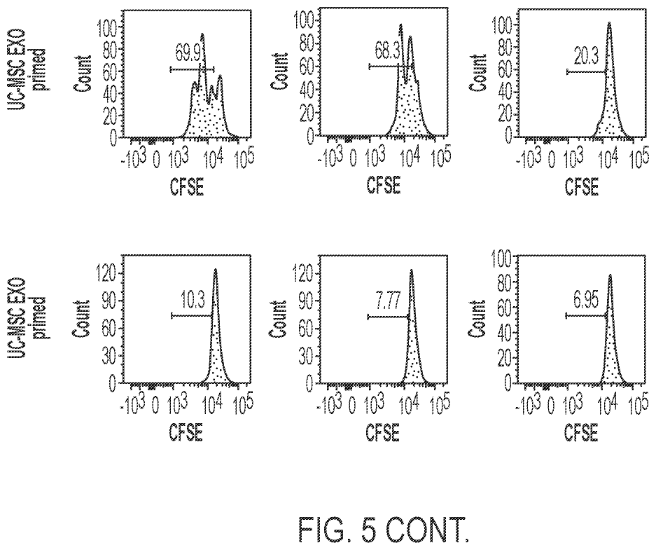

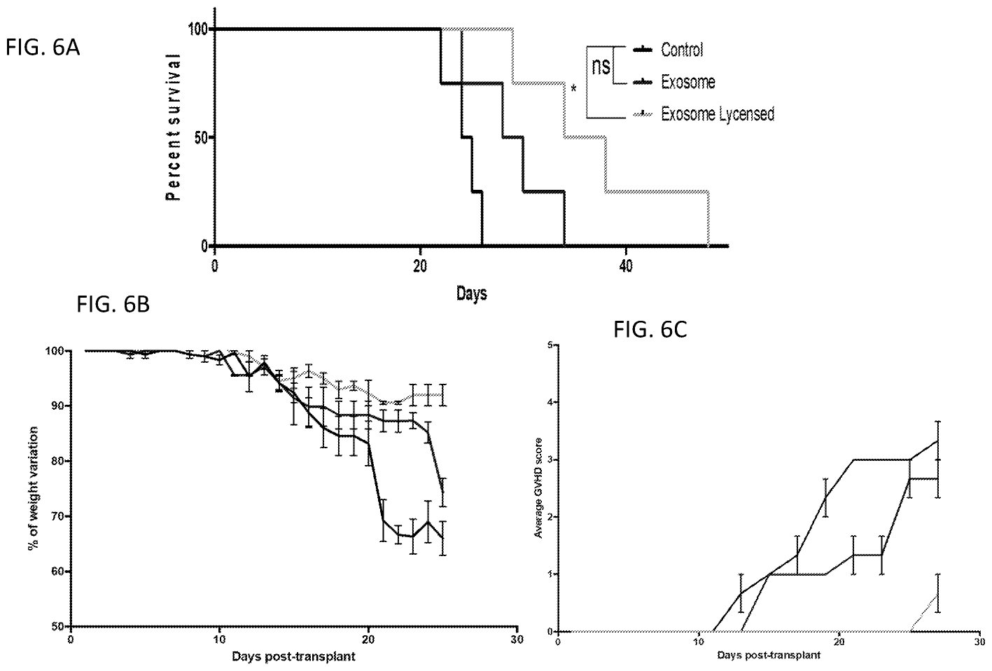

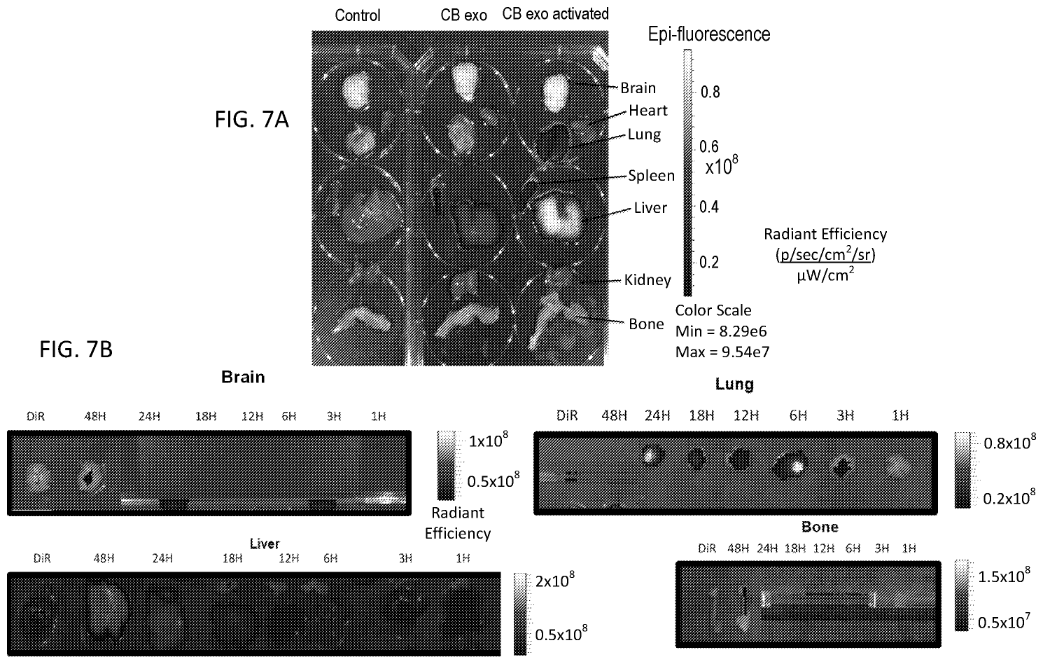

The CBt-MSC-exosomes express several immunomodulatory factors including HLA-G, PD-L1, IL-10, TGF-β, and PD-L2 (FIG. 3), and exhibit high levels of T cell immunosuppression in vitro in a dose-dependent manner (FIG. 4). Importantly, the primed CBt-MSC-derived exosomes exhibit superior control of T cell proliferation in vitro when compared to unprimed CBt-exosomes (FIG. 5). Additionally, the primed CBt-MSC-exosomes exhibit efficient control of GVHD in a xenograft mouse model (FIG. 6). The primed CBt-MSC-exosomes have a higher capacity to migrate to target tissues when compared to their unprimed counterparts (FIG. 7). Finally, these exosome preparations may be efficiently genetically modified by electroporation, viral or other methods to be used as carriers for therapeutic genes or drugs.

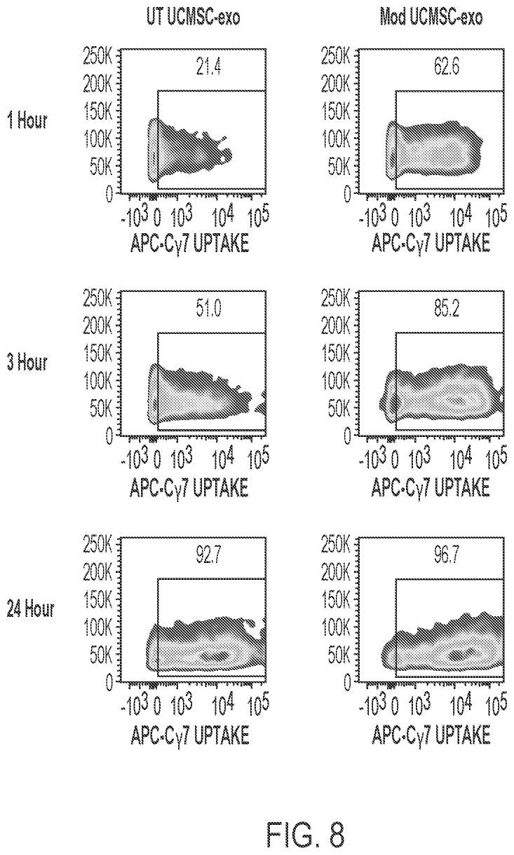

The CBt derived exosomes can be loaded with therapeutic cargo, such as miRNA, siRNA, proteins, and/or drugs (for example) for the treatment of cancer cells, in the autoimmune setting to reduce inflammation, and/or for the repair of damaged vital organs in the regenerative medicine setting. These exosomes can also be used to carry drugs of many types included anti-cancer agent(s), renal drug(s), cardiac drug(s) and pulmonary drug(s) as well as drug(s) for autoimmune disease. Recent reports confirmed that glycan interactions are indeed essential to the uptake of exosomes by recipient cells. FIG. 8 shows that exo-fucosylation of the CBt-MSC exosomes increased the uptake of those exosomes by endothelial and immune cells (FIG. 8), as examples.

### Example 2

### Exo-Fucosylation of Exosomes

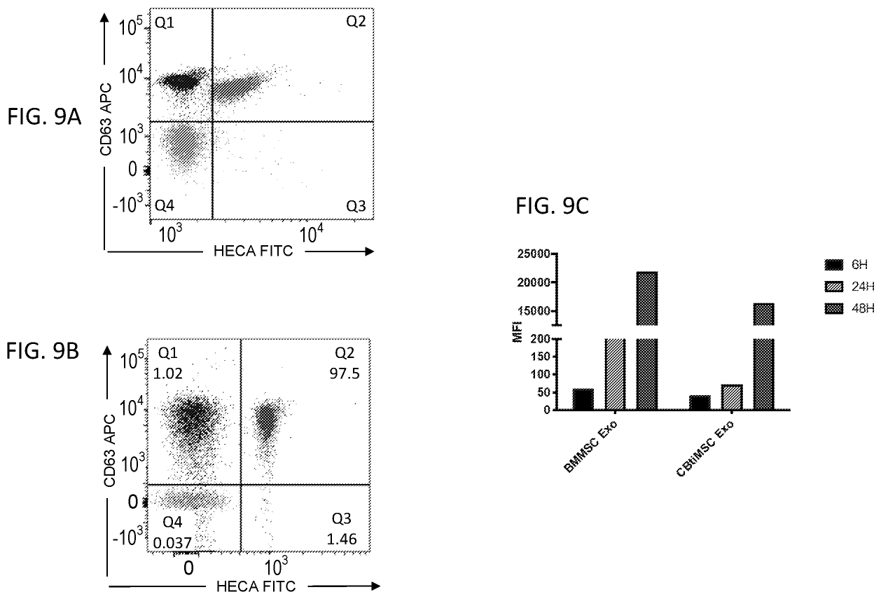

Mesenchymal stromal cells derived from bone marrow (BMMSCs) or cord tissue (CBti MSCs) were transduced with the retroviral construct encoding for the fucosyl transferase (FT)-6 enzyme, and exosomes were isolated by ultracentrifugation from collected supernatant of culture (basal cultures that included alpha-MEM media and glutamine) at serial time points 6H, 24H, and 48H post-transduction to evaluate the expression of sialyl Lewis x (sLe^{X}, determined by HECA-452, a monoclonal antibody (mAb) that recognizes sLe^{x}) on the surface of the collected exosomes (FIG. 9). FIG. 9A shows a representative scatter plot of CD63 expression versus cell-surface fucosylation, sLe^{X} (HECA-452) expression on the surface of exosomes derived from bone marrow MSCs non-transduced (blue), bone marrow MSCs transduced with the enzyme FT-6 (red), using as control the isotype (grey), and then analyzed by flow cytometry. FIG. 9B shows representative scatter plot and mean fluorescent intensity of CD63 expression versus cell-surface fucosylation, SLeX (HECA-452) expression on the surface of exosomes derived from cord blood tissue MSCs non-transduced (blue), bone marrow MSCs transduced with the enzyme FT-6 (red), using as control the isotype (grey), and then analyzed by flow cytometry. Similar results were observed in the case of the mean fluorescent intensity (MFI) of fucosylated exosomes from both BMMSCs and CBtiMSCs collected at 48 hours post-transduction, in comparison with earlier time points. FIG. 9C shows the bar graph of the mean fluorescent intensity (MFI) of FT6-transduced BMMSC-derived exosomes (left) and CbtiMSCs-derived exosomes collected at 6H, 24H, and 48H after transduction. For both BM and CBti exosome sources, the maximal transduction efficiency of MSC-exosomes was observed at 48H in comparison with the exosomes from nontransduced MSCs, in comparison with the appropriate isotype.

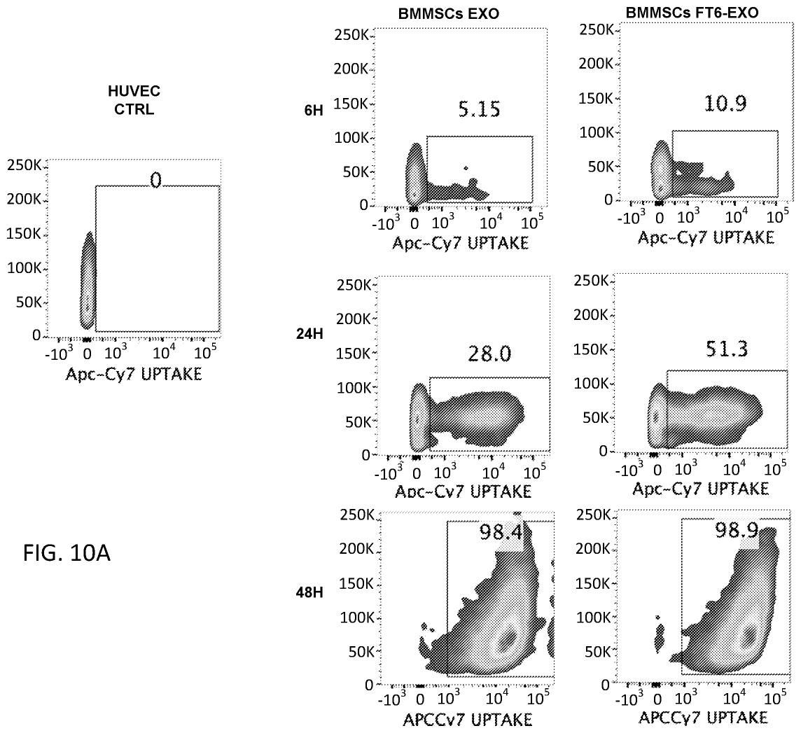

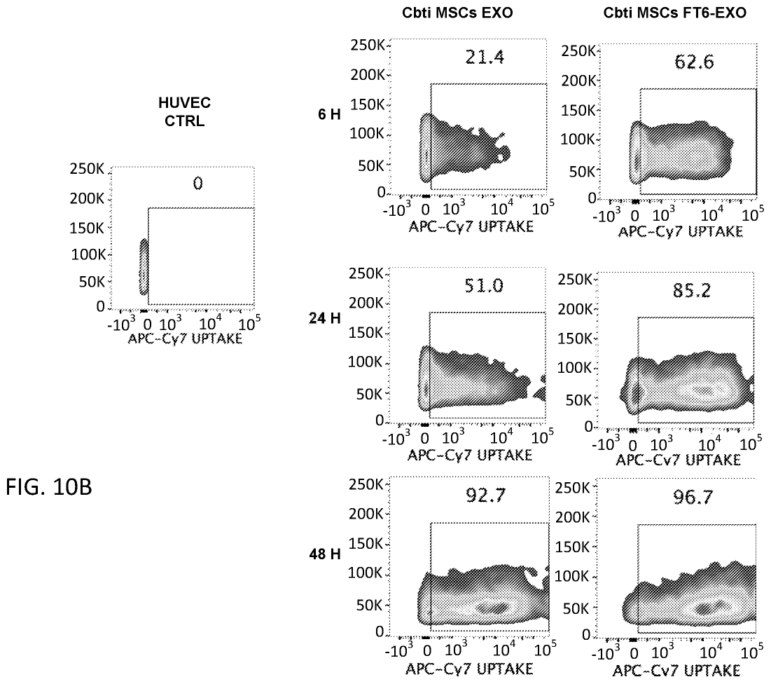

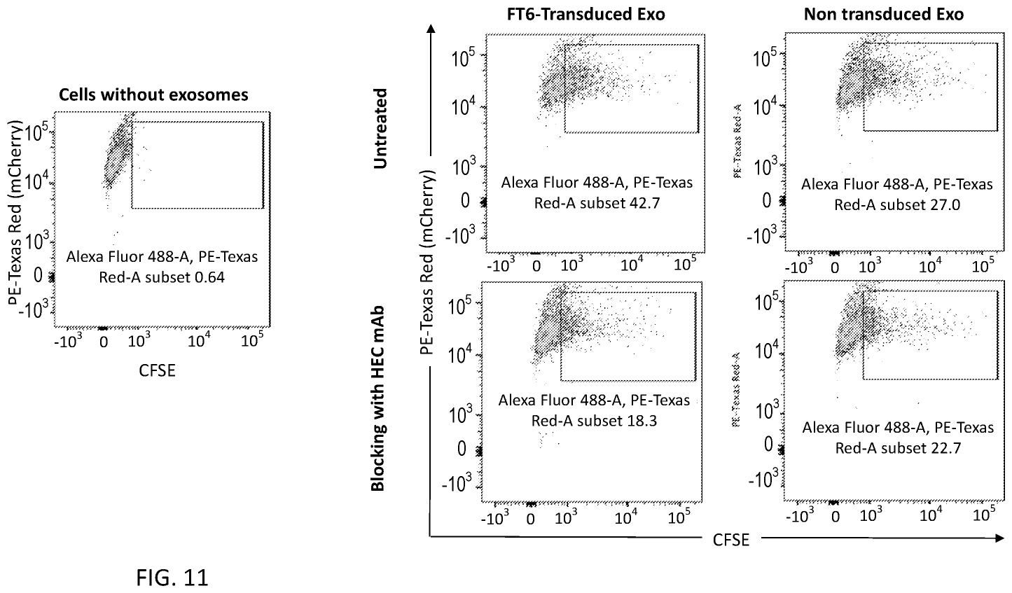

In FIG. 10, there is confirmation that fucosylation of e-selectin ligands on the surface of MSC-exosomes from different sources affect their uptake by Human umbilical vein endothelial cells (HUVEC). Briefly, culture supernatant from FT-6 transduced MSCs and non-transduced MSCs from the two different source (BM and Cbti) were collected after 48 hours, and exosomes were isolated by sequential ultracentrifugation at 100,000 G for 4 hours. Isolated exosomes were labeled using DiR-label, counted by NTA, and added to HUVEC culture. After 6H, 24H, and 48H of coculture, HUVEC cells were trypsinized, and uptake of pre-labeled exosomes from each condition was evaluated by flow cytometry by the detection of fluorescent DiR on the HUVEC. Modification of sLe^{X} expression on the surface of MSC-exosomes by different sources increase their uptake at early time points 6H and 24H. Moreover, in FIG. 11 the increase shown of the uptake of FT-6 transduced MSC-exosomes by normal and tumor cells lines in at least some embodiments is partially attributed to the presence of sLeX residues in their surface. Using glioblastoma cell line GSC 8-11 mCherry, the effect of their uptake was evaluated by blocking sLe^{X} residues in the surface of exosomes isolated from FT-6 transduced CBtiMSCs. Briefly, isolated and CFSE-prelabeled exosomes from both non-transduced and FT-6 transduced CBtiMSCs were incubated with GSC 8-11 for 1 hour. In some studies, HECA-452 mAb were added to the culture in order to block the sLe^{X} residues (FIG. 11), and uptake of CFSE-labeled exosomes was evaluated by flow cytometry by the cells double positive for mCherry (PE) and CFSE (FITC). Blocking of sLe^{X} residues on the surface of FT-6 transduced exosomes by mAb HECA-452 drastically reduced their uptake, in comparison with exosomes from untreated FT6-transduced MSC-exosomes. However, no effect was observed when non-transduced exosomes were evaluated using similar conditions, indicating that the increase of the uptake FT-6 transduced exosomes by tumor cells could be mediated by sLe^{X} residues in their surface (FIG. 11).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of producing exosomes from mesenchymal stromal cells (MSCs), comprising the steps of:
(a) culturing MSCs in the presence of an effective amount of interferon (IFN)γ, tumor necrosis factor (TNF)α, interleukin (IL)-1β, and IL-17; and
(b) collecting the exosomes from the culture.

2. The method of claim 1, wherein the culturing step occurs for at least 18 hours.

3. The method of claim 1 or 2, wherein the culturing step occurs for 18-24 hours.

4. The method of any one of claims 1-3, wherein the collecting step occurs once or multiple times.

5. The method of claim 4, wherein when the collecting step occurs multiple times, the duration between collecting steps is about 1 day, 2 days, 3 days, 4 days, or longer.

6. The method of claim 4 or 5, wherein exosomes collected at different times comprise substantially the same genotype and/or phenotype.

7. The method of any one of claims 1-6, wherein the exosomes comprise higher levels of one or more immunosuppressive factors compared to exosomes produced from culture that does not comprise IFNγ, TNFα, IL-1β, and IL-17.

8. The method of any one of claims 1-7, wherein the exosomes comprise HLA-G, PD-L1, IL-10, TGF-β, IDO, and PD-L2.

9. The method of claim 8, wherein the exosomes comprise higher levels of one or more of HLA-G, PD-L1, IL-10, TGF-β, IDO, and PD-L2 compared to exosomes produced from culture that does not comprise IFNγ, TNFα, IL-1β, and IL-17.

10. The exosomes of any one of claims 1-9, wherein the exosomes comprise the markers CD9, CD63, CD47, and/or CD81.

11. The method of any one of claims 1-9, wherein the culturing step occurs in the presence of specific concentrations or conditions of CO₂, O₂ and nitrogen.

12. The method of claim 11, wherein the concentration of CO₂ is 5%.

13. The method of claim 11 or 12, wherein the concentration of O₂ is 20%.

14. The method of any one of claims 11-13, wherein the culturing step occurs under conditions balanced with nitrogen.

15. The method of any one of claims 1-14, wherein the MSCs are from umbilical cord tissue, bone marrow, adipose tissue, dental tissue, placental tissue, or a mixture thereof.

16. The method of any one of claims 1-15, wherein the exosomes have enhanced control of T cell proliferation compared to exosomes produced from culture that does not comprise IFNγ, TNFα, IL-1β, and IL-17.

17. The method of any one of claims 1-16, wherein the method occurs in an automated system.

18. The method of claim 17, wherein system is configured to comprise continuous perfusion of medium through at least part of the system.

19. The method of claim 17 or 18, wherein the system is closed or semi-closed.

20. The method of any one of claims 1-19, wherein the method occurs in a bioreactor.

21. The method of claim 20, wherein the bioreactor comprises multiple hollow fibers.

22. The method of claim 20, wherein one or more surfaces inside the bioreactor are modified to allow adherence of cells.

23. The method of claim 22, wherein the one or more surfaces inside the bioreactor are modified to comprise one or more extracellular matrix proteins.

24. The method of claim 23, wherein the extracellular matrix protein is fibronectin.

25. The method of any one of claims 17-24, further comprising the step of extracting a sample from the system.

26. The method of claim 25, wherein the sample is tested for one or more characteristics of the exosomes.

27. The method of any one of claims 1-26, wherein step (b) utilizes media that lacks platelet lysate.

28. The method of any one of claims 1-27, wherein step (b) utilizes media that comprises L-alanyl-L-glutamine dipeptide.

29. The method of any one of claims 1-28, wherein the culture in step (a) further comprises media that comprises L-alanyl-L-glutamine dipeptide.

30. The method of any one of claims 1-29, wherein the culture in step (a) further comprises alpha MEM media, heparin, human platelet lysate and L-alanyl-L-glutamine dipeptide.

31. The method of any one of claims 1-30, wherein steps (a) and (b) occur more than once.

32. The method of any one of claims 1-31, wherein steps (a) and (b) occur 2, 3, 4, 5, 6, 7, 8, 9, 10, or more times.

33. The method of any one of claims 1-32, wherein step (b) occurs more than once and the collecting occurs in intervals of about 48 hours.

34. The method of any one of claims 1-33, further comprising the step of delivering an effective amount of the exosomes to an individual in need thereof.

35. The method of claim 34, wherein following delivery to an individual in need thereof, the exosomes have enhanced migration to peripheral tissue compared to exosomes produced from culture that does not comprise IFNγ, TNFα, IL-1β, and IL-17.

36. The method of claim 35, wherein the peripheral tissue is brain, bone marrow, kidney, spleen, or a combination thereof.

37. The method of any one of claims 34-36, wherein the exosomes directly or indirectly regulate an innate immune response or adaptive immune response in the individual in need thereof.

38. The method of any one of claims 34-37, wherein the individual in need thereof has an immune disorder, cancer, heart disease, kidney disease, lung disease, liver disease, infection, or a combination thereof.

39. The method of claim 38, wherein the immune disorder is an autoimmune disorder or an alloimmune disorder.

40. The method of claim 38 or 39, wherein the immune disorder is graft-versus-host disease.

41. The method of any one of claims 34-40, wherein the exosomes are modified before delivery to the individual in need thereof.

42. The method of claim 41, wherein the exosomes are exo-fucosylated before delivery to an individual in need thereof.

43. The method of any one of claims 1-42, wherein the exosomes are loaded to comprise one or more therapeutic agents.

44. The method of claim 43, wherein the exosomes are loaded by a vector, electroporation, transfection, using a cationic liposome transfection agent, or a combination thereof.

45. The method of claim 43 or 44, wherein the one or more therapeutic agents is miRNA, siRNA, shRNA, protein, peptides, drug, lipids, DNA, RNA, or a combination thereof.

46. The method of claim 45, wherein the protein comprises an antibody or antibody fragment.

47. The method of any one of claims 1-46, wherein the exosomes are transduced or transfected with a fucosyl transferase.

48. Exosomes produced from any one of the methods of claims 1-47.

49. A composition comprising the exosomes of claim 48.

50. A pharmaceutical composition comprising the exosomes of claim 48.

51. The pharmaceutical composition of claim 50, further comprising one or more additional therapeutic agents.

52. A method of treating an individual for an immune disorder, cancer, heart disease, kidney disease, lung disease, liver disease, infection, or a combination thereof, comprising the step of administering to the individual a therapeutically effective amount of exosomes produced by the method of any one of claims 1-47.

53. The method of claim 52, wherein the immune disorder is an alloimmune disorder or an autoimmune disorder.

54. The method of claim 52 or 53, further comprising administering to the individual a second therapy for the respective immune disorder, cancer, heart disease, kidney disease, lung disease, liver disease, infection, or a combination thereof.

55. The method of any one of claims 52-54, wherein the MSCs are autologous or allogeneic with respect to the individual.

56. The method of any one of claims 52-55 wherein the exosomes are administered via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir.

57. The method of any one of claims 52-56, wherein the exosomes are administered in conjunction with at least one additional therapeutic agent.
